(19) United States
(12) Patent Application Publication
(10) Pub. No.: US 2024/0316346 A1
(43) Pub. Date: Sep. 26, 2024

(54) SYSTEMS AND METHODS FOR MOVING STIMULATION USING ANATOMICAL DIRECTIONAL CONTROLS

(71) Applicant: Boston Scientific Neuromodulation Corporation, Valencia, CA (US)

(72) Inventors: Chirag Shah, Valencia, CA (US); Peter J. Yoo, Burbank, CA (US)

(21) Appl. No.: 18/610,841

(22) Filed: Mar. 20, 2024

Related U.S. Application Data
(60) Provisional application No. 63/453,884, filed on Mar. 22, 2023.

(51) Int. Cl. A61N 1/36; A61N 1/05; A61N 1/372

(52) U.S. Cl. CPC A61N 1/36185; A61N 1/05; A61N 1/37241; A61N 1/37247; A61N 1/37264

(57) ABSTRACT

A method for modifying stimulation includes providing stimulation to the patient by the stimulation device through at least one of the electrodes or optical emitters of a stimulation lead according to an initial set of stimulation parameters; receiving, at a programming device, a user input to move the stimulation in a selected anatomically-defined direction; determining, by the programming device or the stimulation device, a modified set of stimulation parameters that moves the stimulation in the selected anatomically-defined direction; and providing stimulation to the patient by the stimulation device through at least one of the electrodes or optical emitters of a stimulation lead according to the modified set of stimulation parameters.

### CROSS-REFERENCE TO RELATED APPLICATIONS

This application claims the benefit under 35 U.S.C. § 119(e) of U.S. Provisional Patent Application Ser. No. 63/453,884, filed Mar. 22, 2023, which is incorporated herein by reference.

### FIELD

The present disclosure is directed to the area of methods and systems for stimulation including electrical stimulation. The present disclosure is also directed to methods and systems for moving stimulation using anatomical directional controls.

### BACKGROUND

Implantable electrical stimulation systems have proven therapeutic in a variety of diseases and disorders. For example, deep brain stimulation systems have been used as a therapeutic modality for the treatment of Parkinson's disease, essential tremor, and the like. Stimulators have been developed to provide therapy for a variety of treatments. A stimulator can include an implantable pulse generator (IPG), one or more leads, and an array of stimulator electrodes on each lead. The stimulator electrodes are in contact with or near the neurons, nerves, muscles, or other tissue to be stimulated. The pulse generator in the IPG generates electrical pulses that are delivered by the electrodes to body tissue. Optical stimulation systems can also be used.

Implantable medical devices (IMDs), including IPGs, typically have the capability to communicate data with an external device, such as a clinician programmer or a remote control, via a radio-frequency telemetry link or other wireless communication method. The clinician programmer can program the operating parameters of the implanted medical device. The remote control can switch programs. Modern implantable devices also include the capability for bidirectional communication so that information can be transmitted to the clinician programmer or remote control from the implanted device.

### BRIEF SUMMARY

One aspect is a method for modifying stimulation by a stimulation system that includes a stimulation device and a stimulation lead coupled to the stimulation device and having a distal end portion implanted in a patient and a plurality of electrodes, optical emitters, or any combination thereof disposed along the distal end portion, wherein the distal end portion is not parallel to at least one of a sagittal plane or a coronal plane of the patient. The method includes providing stimulation to the patient by the stimulation device through at least one of the electrodes or optical emitters of the stimulation lead according to an initial set of stimulation parameters; receiving, at a programming device, a user input to move the stimulation in a selected anatomically-defined direction; determining, by the programming device or the stimulation device, a modified set of stimulation parameters that moves the stimulation in the selected anatomically-defined direction; and providing stimulation to the patient by the stimulation device through at least one of the electrodes or optical emitters of the stimulation lead according to the modified set of stimulation parameters.

In at least some aspects, the anatomically-defined direction is selected from anterior, posterior, superior, inferior, lateral, medial, or any combination thereof. In at least some aspects, the method further includes obtaining a trajectory of the distal end portion of the stimulation lead. In at least some aspects, obtaining the trajectory includes receiving, at the programming device, a user input of the trajectory. In at least some aspects, obtaining the trajectory includes determining an actual or estimated trajectory from imaging.

In at least some aspects, the method further includes obtaining a rotational orientation of the stimulation lead. In at least some aspects, obtaining the rotational orientation includes observing an orientation marker of the stimulation lead or estimating the rotational orientation from responses to stimulation. In at least some aspects, obtaining the rotational orientation includes determining the rotational orientation from imaging of the stimulation lead.

In at least some aspects, determining the modified set of stimulation parameters including transforming the selected anatomically-defined direction into a lead coordinate system or at least one lead direction. In at least some aspects, determining the modified set of stimulation parameters includes determining the modified set of stimulation parameters using a predefined algorithm with the selected anatomically-defined direction being an input to the algorithm. In at least some aspects, the programming device includes a control for switching between user input controls for moving stimulation along anatomically-defined directions and user input controls for moving stimulation along lead directions.

Another aspect is a programming device for modifying stimulation by a stimulation system including a stimulation device and a stimulation lead coupled to the stimulation device and having a distal end portion implanted in a patient and a plurality of electrodes or optical emitters disposed along the distal end portion, wherein the distal end portion is not parallel to at least one of a sagittal plane or a coronal plane of the patient. The system includes at least one processor configured to perform actions, the actions including: receiving a user input to move stimulation in a selected anatomically-defined direction; determining a modified set of stimulation parameters that moves the stimulation in the selected anatomically-defined direction; and transmitting the modified set of stimulation parameters to the stimulation device.

A further aspect is a non-transient computer readable medium having instructions for performing actions stored thereon for modifying stimulation by a stimulation system including a stimulation device and a stimulation lead coupled to the stimulation device and having a distal end portion implanted in a patient and a plurality of electrodes or optical emitters disposed along the distal end portion, wherein the distal end portion is not parallel to at least one of a sagittal plane or a coronal plane of the patient. The actions including: receiving a user input to move stimulation in a selected anatomically-defined direction; determining a modified set of stimulation parameters that moves the stimulation in the selected anatomically-defined direction; and transmitting the modified set of stimulation parameters to the stimulation device.

In at least some aspects, the anatomically-defined direction is selected from anterior, posterior, superior, inferior, lateral, medial, or any combination thereof. In at least some aspects, the method or actions further include obtaining a trajectory of the distal end portion of the stimulation lead. In at least some aspects, obtaining the trajectory includes receiving, at the programming device, a user input of the trajectory. In at least some aspects, obtaining the trajectory includes determining an actual or estimated trajectory from imaging.

In at least some aspects, the method or actions further include obtaining a rotational orientation of the stimulation lead. In at least some aspects, obtaining the rotational orientation includes observing an orientation marker of the stimulation lead or estimating the rotational orientation from responses to stimulation. In at least some aspects, obtaining the rotational orientation includes determining the rotational orientation from imaging of the stimulation lead.

In at least some aspects, determining the modified set of stimulation parameters including transforming the selected anatomically-defined direction into a lead coordinate system or at least one lead direction. In at least some aspects, determining the modified set of stimulation parameters includes determining the modified set of stimulation parameters using a predefined algorithm with the selected anatomically-defined direction being an input to the algorithm. In at least some aspects, the programming device includes a control for switching between user input controls for moving stimulation along anatomically-defined directions and user input controls for moving stimulation along lead directions.

Yet another embodiments is a stimulation system that includes any of the programming devices described above, a stimulation lead including electrodes, optical emitters, or any combination thereof; and a stimulation device coupled or coupleable to the stimulation lead, wherein the stimulation device includes a processor configured to perform actions, the actions including providing stimulation to the patient through at least one of the electrodes or optical emitters of the stimulation lead according to an initial set of stimulation parameters, and after the modified set of stimulation parameters is transmitted to the stimulation device, providing stimulation to the patient through at least one of the electrodes or optical emitters of the stimulation lead according to the modified set of stimulation parameters.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes one or more leads that can be coupled to an IPG;

FIG. 2 is a block diagram of elements of an electrical stimulation system;

FIG. 3A is a schematic perspective view of a distal portion of one embodiment of an electrical stimulation lead with segmented electrodes;

FIG. 3B is a schematic perspective view of a distal portion of another embodiment of an electrical stimulation lead with segmented electrodes;

FIG. 3C is a schematic perspective view of a distal portion of a third embodiment of an electrical stimulation lead with segmented electrodes;

FIG. 3D is a schematic perspective view of a distal portion of a fourth embodiment of an electrical stimulation lead with segmented electrodes;

FIG. 3E is a schematic perspective view of a distal portion of a fifth embodiment of an electrical stimulation lead with segmented electrodes;

FIG. 4 is a schematic perspective view of a distal portion of a sixth embodiment of an electrical stimulation lead with segmented electrodes and an orientation marker;

FIG. 5 is a schematic perspective view of a distal portion of the electrical stimulation lead of FIG. 3B implanted at an angle relative to both the coronal and sagittal planes of a patient;

FIG. 6 is a schematic diagram of one embodiment of an interface for obtaining a trajectory and optional rotation orientation of a stimulation lead implanted in a patient;

FIG. 7 is a schematic diagram of one embodiment of an interface for programming a stimulation system including controls for moving stimulation along anatomically-defined directions; and

FIG. 8 is a flowchart of one embodiment of a method for moving stimulation using anatomically-defined directions.

### DETAILED DESCRIPTION

The present disclosure is directed to the area of methods and systems for stimulation including electrical stimulation. The present disclosure is also directed to methods and systems for moving stimulation using anatomical directional controls.

Implantable electrical stimulation systems and devices are used herein to exemplify the inventions, but it will be understood that these inventions can be utilized with other stimulation or modulation systems and devices, such as optical or electrical/optical stimulation or modulation systems. Examples of implantable electrical stimulation systems include, but are not limited to, a least one lead with one or more electrodes disposed along a distal end of the lead and one or more terminals disposed along the one or more proximal ends of the lead. Examples of electrical stimulation systems with leads are found in, for example, U.S. Pat. Nos. 6,181,969; 6,295,944; 6,391,985; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,831,742; 8,688,235; 8,175,710; 8,224,450; 8,271,094; 8,295,944; 8,364,278; and 8,391,985; U.S. Patent Application Publications Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2011/0005069; 2010/0268298; 2011/0130817; 2011/0130818; 2011/0078900; 2011/0238129; 2011/0313500; 2012/0016378; 2012/0046710; 2012/0165911; 2012/0197375; 2012/0203316; 2012/0203320; and 2012/0203321, all of which are incorporated by reference in their entireties. Examples of optical stimulation or modulation systems or electrical/optical stimulation systems, which include one or more optical emitters in addition to, or as an alternative to, electrodes, are found in U.S. Pat. Nos. 9,415,154; 10,335,607; 10,625,072; and 10,814,140 and U.S. Patent Application Publications Nos. 2013/0317572; 2013/0317573; 2017/0259078; 2017/0225007; 2018/0110971; 2018/0369606; 2018/0369607; 2019/0209849; 2019/0209834; 2020/0094047; 2020/0155584; 2020/0376262; 2021/0008388; 2021/0008389; 2021/0016111; and 2022/0072329, all of which are incorporated by reference in their entireties.

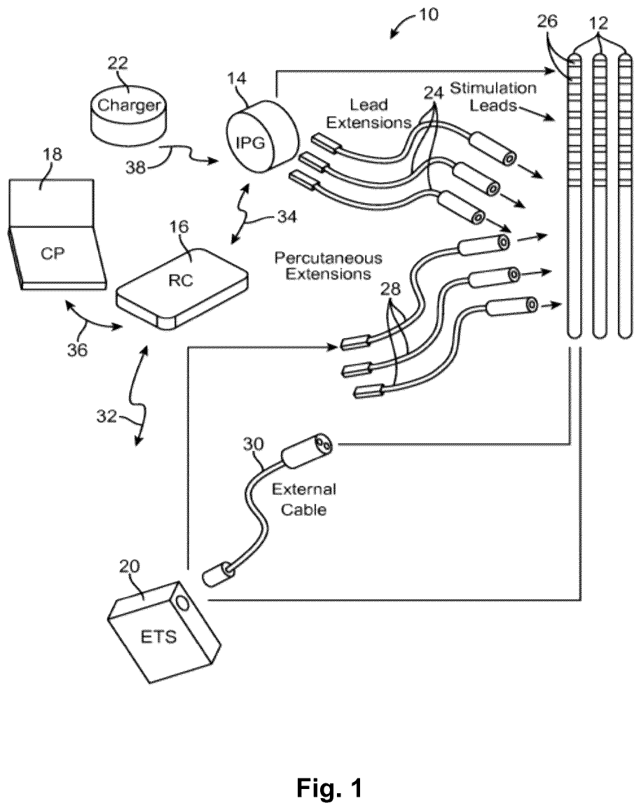

Turning to FIG. 1, one embodiment of an electrical stimulation system 10 includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22. The IPG and ETS are examples of control modules for the electrical stimulation system.

The IPG 14 is physically connected, optionally via one or more lead extensions 24, to the stimulation lead(s) 12. Each lead carries multiple electrodes 26 arranged in an array along a distal end portion of the lead. A lead 12 can have any suitable number of electrodes including, but not limited to, 1, 2, 4, 8, 10, 12, 16, 20, 24, 32, 40, 50, or 64 electrodes. In at least some embodiments, each lead also carries multiple terminals (not shown) arranged in an array along a proximal end portion of the lead and coupled to the electrodes.

The IPG 14 includes pulse generation circuitry that delivers electrical stimulation energy in the form of, for example, a pulsed electrical waveform (i.e., a temporal series of electrical pulses) to one or more electrodes of the electrode array 26 in accordance with a set of stimulation parameters. The implantable pulse generator can be implanted into a patient's body, for example, below the patient's clavicle area or within the patient's abdominal cavity or at any other suitable site. The implantable pulse generator 14 can have multiple stimulation channels which may be independently programmable to control the magnitude of the current stimulus from each channel. In some embodiments, the implantable pulse generator 14 can have any suitable number of stimulation channels including, but not limited to, 4, 6, 8, 12, 16, 32, or more stimulation channels. The implantable pulse generator 14 can have one, two, three, four, or more connector ports, for receiving the terminals of the leads and/or lead extensions.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni- or bi-directional communications link 34. Such communication or control allows the IPG 14, for example, to be turned on or off and to be programmed with different stimulation parameter sets. The IPG 14 may also be operated to modify the programmed stimulation parameters to actively control the characteristics of the electrical stimulation energy output by the IPG 14. In at least some embodiments, the CP 18 (or RC 16 or other programming device) allows a user, such as a clinician, the ability to program stimulation parameters for the IPG 14 and ETS 20 in the operating room and in follow-up sessions. Alternately, or additionally, in at least some embodiments, stimulation parameters can be programed via wireless communications (e.g., Bluetooth) between the RC 16 (or other external device such as a hand-held electronic device like a mobile phone, tablet, or the like) and the IPG 14.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). In at least some embodiments, the stimulation parameters provided by the CP 18 are also used to program the RC 16, so that the stimulation parameters can be subsequently modified by operation of the RC 16 in a stand-alone mode (i.e., without the assistance of the CP 18). The CP 18 or RC 16 can be any suitable device including, but not limited to, a computer or other computing device, laptop, mobile device (for example, a mobile phone or tablet), or the like or any combination thereof. The CP 18 or RC 16 can include software applications for interacting with the IPG 14 or ETS 20 and for programming the IPG 14 or ETS 20.

Additional examples of the RC 16, CP 18, ETS 20, and external charger 22 can be found in the references cited herein as well as U.S. Pat. Nos. 6,895,280; 6,181,969; 6,516,227; 6,609,029; 6,609,032; 6,741,892; 7,949,395; 7,244,150; 7,672,734; and 7,761,165; 7,974,706; 8,175,710; 8,224,450; and 8,364,278; and U.S. Patent Application Publication No. 2007/0150036, all of which are incorporated herein by reference in their entireties.

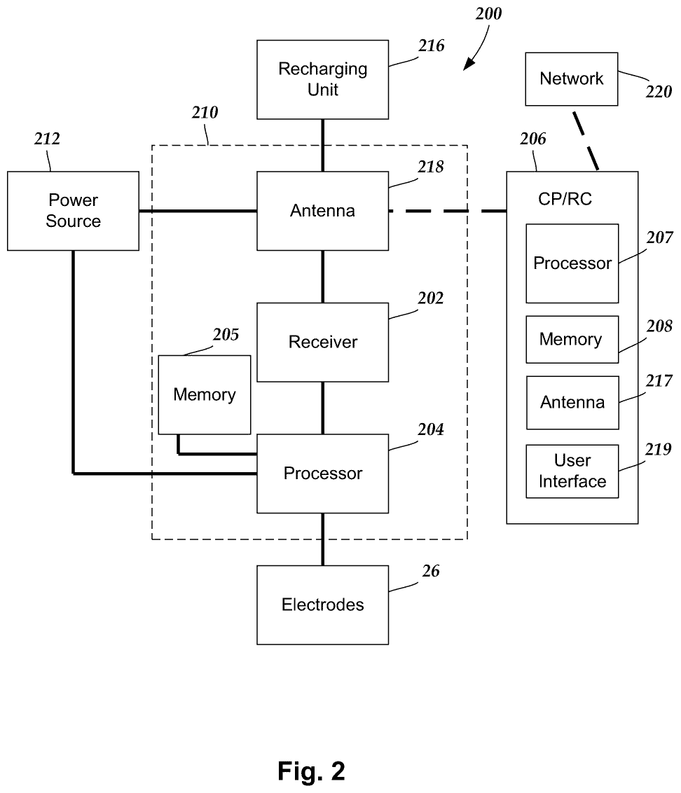

FIG. 2 is a schematic overview of one embodiment of components of an electrical stimulation system 200 including an electronic subassembly 210 disposed within an IPG 14 (FIG. 1). It will be understood that the electrical stimulation system can include more, fewer, or different components and can have a variety of different configurations including those configurations disclosed in the stimulator references cited herein.

The IPG 14 (FIG. 1) can include, for example, a power source 212, antenna 218, receiver 202, processor 204, and memory 205. Some of the components (for example, power source 212, antenna 218, receiver 202, processor 204, and memory 205) of the electrical stimulation system can be positioned on one or more circuit boards or similar carriers within a sealed housing of the IPG 14 (FIG. 1), if desired. Unless indicated otherwise, the term “processor” refers to both embodiments with a single processor and embodiments with multiple processors.

An external device, such as a CP or RC 206, can include a processor 207, memory 208, an antenna 217, and a user interface 219. The user interface 219 can include, but is not limited to, a display screen on which a digital user interface can be displayed and any suitable user input device, such as a keyboard, touchscreen, mouse, track ball, or the like or any combination thereof.

Any power source 212 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference in its entirety.

As another alternative, power can be supplied by an external power source through inductive coupling via the antenna 218 or a secondary antenna. The external power source can be in a device that is mounted on the skin of the user or in a unit that is provided near the user on a permanent or periodic basis.

If the power source 212 is a rechargeable battery, the battery may be recharged using the antenna 218, if desired. Power can be provided to the battery for recharging by inductively coupling the battery through the antenna to a recharging unit 216 external to the user. Examples of such arrangements can be found in the references identified above.

In one embodiment, electrical current is emitted by the electrodes 26 on the lead body to stimulate nerve fibers, muscle fibers, or other body tissues near the electrical stimulation system. A processor 204 is generally included to control the timing and electrical characteristics of the electrical stimulation system. For example, the processor 204 can, if desired, control one or more of the timing, frequency, amplitude, width, and waveform of the pulses. In addition, the processor 204 can select which electrodes can be used to provide stimulation, if desired. In some embodiments, the processor 204 may select which electrode(s) are cathodes and which electrode(s) are anodes. In some embodiments, the processor 204 may be used to identify which electrodes provide the most useful stimulation of the desired tissue. Instructions for the processor 204 can be stored on the memory 205. Instructions for the processor 207 can be stored on the memory 208.

Any processor 204 can be used for the IPG and can be as simple as an electronic device that, for example, produces pulses at a regular interval or the processor can be capable of receiving and interpreting instructions from the CP/RC 206 (such as CP 18 or RC 16 of FIG. 1) that, for example, allows modification of pulse characteristics. In the illustrated embodiment, the processor 204 is coupled to a receiver 202 which, in turn, is coupled to the antenna 218. This allows the processor 204 to receive instructions from an external source to, for example, direct the pulse characteristics and the selection of electrodes, if desired. Any suitable processor 207 can be used for the CP/RC 206.

Any suitable memory 205, 208 can be used including computer-readable storage media may include, but is not limited to, volatile, nonvolatile, non-transitory, removable, and non-removable media implemented in any method or technology for storage of information, such as computer readable instructions, data structures, program modules, or other data. Examples of computer-readable storage media include, but are not limited to, RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a processor.

In one embodiment, the antenna 218 is capable of receiving signals (e.g., RF signals) from an antenna 217 of a CP/RC 206 (see, CP 18 or RC 16 of FIG. 1) which is programmed or otherwise operated by a user. The signals sent to the processor 204 via the antenna 218 and receiver 202 can be used to modify or otherwise direct the operation of the electrical stimulation system. For example, the signals may be used to modify the pulses of the electrical stimulation system such as modifying one or more of pulse width, pulse frequency, pulse waveform, and pulse amplitude. The signals may also direct the electrical stimulation system 200 to cease operation, to start operation, to start signal acquisition, to stop signal acquisition, to start charging the battery, or to stop charging the battery. In other embodiments, the stimulation system does not include an antenna 218 or receiver 202 and the processor 204 operates as programmed.

Optionally, the electrical stimulation system 200 may include a transmitter (not shown) coupled to the processor 204 and the antenna 218 for transmitting signals back to the CP/RC 206 or another unit capable of receiving the signals. For example, the electrical stimulation system 200 may transmit signals indicating whether the electrical stimulation system 200 is operating properly or not or indicating when the battery needs to be charged or the level of charge remaining in the battery. The processor 204 may also be capable of transmitting information about the pulse characteristics so that a user or clinician can determine or verify the characteristics.

Transmission of signals can occur using any suitable method, technique, or platform including, but not limited to, inductive transmission, radiofrequency transmission, Bluetooth™, Wi-Fi, cellular transmission, near field transmission, infrared transmission, or the like or any combination thereof. In addition, the IPG 14 can be wirelessly coupled to the RC 16 or CP 18 using any suitable arrangement include direct transmission or transmission through a network, such as a local area network, wide area network, the Internet, or the like or any combination thereof. The CP 18 or RC 16 may also be capable of coupling to, and sending data or other information to, a network 220, such as a local area network, wide area network, the Internet, or the like or any combination thereof.

At least some of the stimulation electrodes can take the form of segmented electrodes that extend only partially around the perimeter (for example, the circumference) of the lead. These segmented electrodes can be provided in sets of electrodes, with each set having electrodes circumferentially distributed about the lead at a particular longitudinal position.

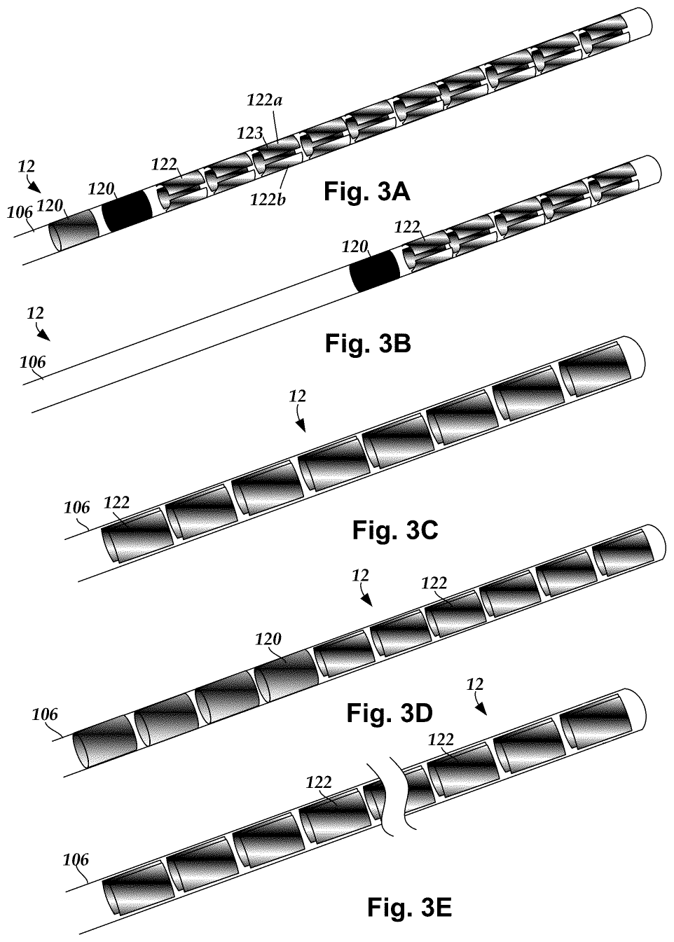

In FIGS. 3A, 3B, and 3D the electrodes on a distal end portion of a lead 12 are shown as including both ring electrodes 120 and segmented electrodes 122. In some embodiments, the electrodes are all segmented electrode 122, as illustrated in FIGS. 3C and 3E. The segmented electrodes 122 of FIG. 3A are in sets of three, where the three segmented electrodes of a particular set are electrically isolated from one another and are circumferentially offset along the lead 12. Any suitable number of segmented electrodes can be formed into a set including, for example, two, three, four, or more segmented electrodes. The lead 12 of FIG. 3A has thirty segmented electrodes 122 (ten sets of three electrodes each) and two ring electrodes 120 for a total of 32 electrodes.

Segmented electrodes can be used to direct stimulus current to one side, or even a portion of one side, of the lead. When segmented electrodes are used in conjunction with an implantable pulse generator that delivers current stimulus, current steering can be achieved to deliver the stimulus more precisely to a position around an axis of the lead (i.e., radial positioning around the axis of the lead). Segmented electrodes may provide for superior current steering than ring electrodes because target structures in deep brain stimulation are not typically symmetric about the axis of the distal electrode array. Instead, a target may be located on one side of a plane running through the axis of the lead. Through the use of a segmented electrode array, current steering can be performed not only along a length of the lead but also around a perimeter of the lead. This provides precise three-dimensional targeting and delivery of the current stimulus to neural target tissue, while potentially avoiding stimulation of other tissue.

FIG. 3A illustrates a 32-electrode lead 12 with a lead body 106 and two ring electrodes 120 proximal to thirty segmented electrodes 122 arranged in ten sets of three segmented electrodes each. In the illustrated embodiments, the ring electrodes 120 are proximal to the segmented electrodes 122. In other embodiments, the ring electrodes 120 can be proximal to, or distal to, or any combination thereof.

Any number of segmented electrodes 122 may be disposed on the lead body including, for example, one, two, three, four, five, six, seven, eight, nine, ten, eleven, twelve, thirteen, fourteen, fifteen, sixteen, twenty, twenty-four, twenty-eight, thirty, thirty-two, or more segmented electrodes 122. It will be understood that any number of segmented electrodes 122 may be disposed along the length of the lead body. A segmented electrode 122 typically extends only 75%, 67%, 60%, 50%, 40%, 33%, 25%, 20%, 17%, 15%, or less around the circumference of the lead.

The segmented electrodes 122 may be grouped into sets of segmented electrodes, where each set is disposed around a circumference of the lead 12 at a particular longitudinal portion of the lead 12. The lead 12 may have any number of segmented electrodes 122 in a given set of segmented electrodes. The lead 12 may have one, two, three, four, five, six, seven, eight, or more segmented electrodes 122 in a given set. The lead 12 may have any number of sets of segmented electrode including, but not limited to, one, two, three, four, five, six, eight, ten, twelve, fifteen, sixteen, twenty, or more sets. The segmented electrodes 122 may be uniform, or vary, in size and shape. In some embodiments, the segmented electrodes 122 are all of the same size, shape, diameter, width or area or any combination thereof. In some embodiments, the segmented electrodes 122 of each circumferential set (or even all segmented electrodes disposed on the lead 12) may be identical in size and shape.

Each set of segmented electrodes 122 may be disposed around the circumference of the lead body to form a substantially cylindrical shape around the lead body. The spacing between individual electrodes of a given set of the segmented electrodes may be the same, or different from, the spacing between individual electrodes of another set of segmented electrodes on the lead 12. In at least some embodiments, equal spaces, gaps or cutouts are disposed between each segmented electrode 122 around the circumference of the lead body. In other embodiments, the spaces, gaps or cutouts between the segmented electrodes 122 may differ in size or shape. In other embodiments, the spaces, gaps, or cutouts between segmented electrodes 122 may be uniform for a particular set of the segmented electrodes 122, or for all sets of the segmented electrodes 122. The sets of segmented electrodes 122 may be positioned in irregular or regular intervals along a length of the lead body.

The electrodes of the lead 12 are typically disposed in, or separated by, a non-conductive, biocompatible material of a lead body 106 including, for example, silicone, polyurethane, and the like or combinations thereof. The lead body 106 may be formed in the desired shape by any process including, for example, extruding, molding (including injection molding), casting, and the like. Electrodes and connecting wires can be disposed onto or within a lead body either prior to or subsequent to a molding or casting process. The non-conductive material typically extends from the distal end of the lead body 106 to the proximal end of the lead body 106.

FIG. 3B to 3E illustrate other embodiments of leads with segmented electrodes 122. FIG. 3B illustrates a sixteen electrode lead 12 having one ring electrode 120 that is proximal to five sets of three segmented electrodes 122 each. FIG. 3C illustrates a sixteen electrode lead 12 having eight sets of two segmented electrodes 122 each. As illustrated in FIG. 3C, an embodiment of a lead 12 does not necessarily include a ring electrode. FIG. 3D illustrates a sixteen electrode lead 12 having four ring electrodes 120 that are proximal to six sets of two segmented electrodes 122 each. FIG. 3E illustrates a thirty-two electrode lead 12 having sixteen sets of two segmented electrodes 122 each (for clarity of illustration, not all of the electrodes are shown). It will be recognized that any other electrode combination of ring electrodes, segmented electrodes, or both types of electrodes can be used.

When the lead 12 includes both ring electrodes 120 and segmented electrodes 122, the ring electrodes 120 and the segmented electrodes 122 may be arranged in any suitable configuration. For example, when the lead 12 includes two or more ring electrodes 120 and one or more sets of segmented electrodes 122, the ring electrodes 120 can flank the one or more sets of segmented electrodes 122. Alternately, the two or more ring electrodes 120 can be disposed proximal to the one or more sets of segmented electrodes 122 or the two or more ring electrodes 120 can be disposed distal to the one or more sets of segmented electrodes 122.

The electrodes 120, 122 may have any suitable longitudinal length including, but not limited to, 2, 3, 4, 4.5, 5, or 6 mm. The longitudinal spacing between adjacent electrodes 120, 122 may be any suitable amount including, but not limited to, 1, 2, or 3 mm, where the spacing is defined as the distance between the nearest edges of two adjacent electrodes. In some embodiments, the spacing is uniform between longitudinally adjacent of electrodes along the length of the lead. In other embodiments, the spacing between longitudinally adjacent electrodes may be different or non-uniform along the length of the lead.

Examples of leads with segmented electrodes include U.S. Patent Application Publications Nos. 2010/0268298; 2011/0005069; 2011/0078900; 2011/0130803; 2011/0130816; 2011/0130817; 2011/0130818; 2011/0078900; 2011/0238129; 2011/0313500; 2012/0016378; 2012/0046710; 2012/0071949; 2012/0165911; 2012/0197375; 2012/0203316; 2012/0203320; 2012/0203321; 2013/0197602; 2013/0261684; 2013/0325091; 2013/0317587; 2014/0039587; 2014/0353001; 2014/0358209; 2014/0358210; 2015/0018915; 2015/0021817; 2015/0045864; 2015/0021817; 2015/0066120; 2013/0197424; 2015/0151113; 2014/0358207; and U.S. Pat. No. 8,483,237, all of which are incorporated herein by reference in their entireties. A lead may also include a tip electrode and examples of leads with tip electrodes include at least some of the previously cited references, as well as U.S. Patent Application Publications Nos. 2014/0296953 and 2014/0343647, all of which are incorporated herein by reference in their entireties. A lead with segmented electrodes may be a directional lead that can provide stimulation in a particular direction using the segmented electrodes.

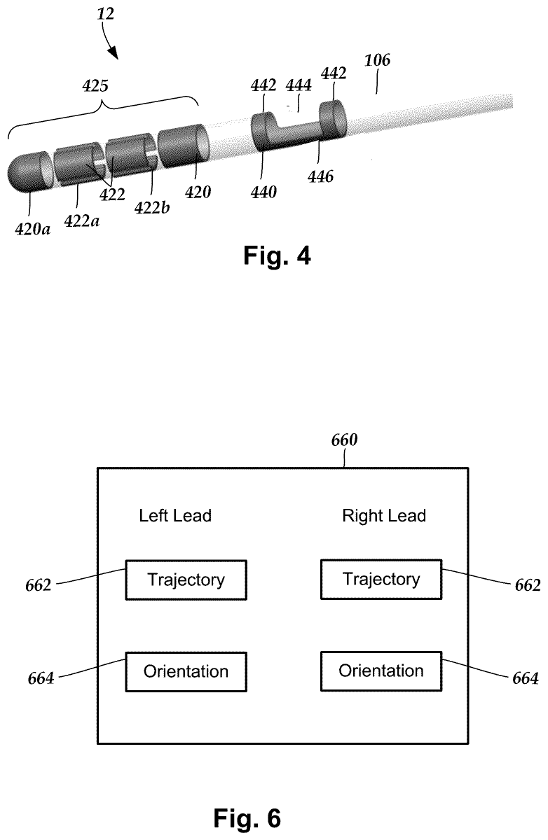

To facilitate radiological identification of rotational orientation, the lead can include a rotationally asymmetric marker made of different material (for example, a conductive material such as metal) from the lead body so that the marker and lead body are radiologically distinguishable. FIG. 4 illustrates one example of a distal portion of a lead 12 with a lead body 106 and electrodes 425 including one or more optional ring electrodes 420, 420a and multiple segmented electrodes 422. The lead 12 also includes a marker 440 that is asymmetrically shaped. The marker 440 is made of a material that is substantially different from the material of the lead body 106, particularly, when viewed using a radiological imaging technique, such as CT imaging, so that the marker is radiologically distinguishable from the lead body. In at least some embodiments, the marker 440 is made of metal (such as a pure metal or an alloy) and, in at least some embodiments, is made of the same material as the electrodes 425.

The marker 440 defines one or more optional rings 442 formed around the entire perimeter of the lead 12, at least one window 444, and a longitudinal band 446 disposed opposite the window. In at least some embodiments, the longitudinal band 446 of the marker 440 extends around no more than 80%, 75%, 67%, 60%, 50%, 40%, 34%, 30%, 25%, or 20% of the perimeter of the lead with the window 444 extending around the remainder of the perimeter. In at least some embodiments, to further facilitate the determination of directionality of the marker 440, the longitudinal band 446 will extend around less than half the perimeter of the lead and may extend around no more than one third or one quarter of the perimeter. In at least some embodiments, the longitudinal band 446 of the marker 440 is aligned with at least one of the segmented electrodes 422 (such as segmented electrodes 422a, 422b in the illustrated embodiment of FIG. 4.) In the illustrated embodiments, the longitudinal band 446 extends between two rings 442.

It will be understood that any other suitable method can be used for determining rotational orientation or stimulation lead placement within the tissue. For example, the physiological response by the patient or patient tissue can be used to predict or estimate rotational orientation or stimulation lead placement. In at least some embodiments, the response to stimulation using different electrodes or optical emitters, as well as a prediction of response by different anatomical structures or regions, can be used to predict or estimate the rotational orientation or placement of the stimulation lead. Examples of systems and methods for estimating a spatial relationship between a stimulation lead and anatomical structures or regions can be found in U.S. Pat. No. 10,716,505, incorporated herein by reference in its entirety.

The region of tissue that is stimulated depends, at least in part, on the selection of electrodes, the distribution of the stimulation amplitude between the electrodes (which can be referred to as “electrode fractionalization”), and the values of other stimulation parameters. As an example, using the stimulation lead of FIG. 3A, selecting one electrode 112a in a set of segmented electrodes 123 for delivery of the stimulation will direct the stimulation to the region of tissue near the electrode 112a. Changing the electrode fractionalization to deliver a portion of the stimulation using electrode 112a and another portion of the stimulation using electrode 112b will expand the circumferential extent of the stimulation toward the tissue near electrode 112b. For example, an electrode fractionalization with 90% of the stimulation amplitude delivered through electrode 112a and 10% of the stimulation amplitude delivered through electrode 112b will extend the circumferential extent of the stimulation toward the tissue near electrode 112b, but the bulk of the stimulation will be directed to the tissue near electrode 112a. Shifting the electrode fractionalization to 50% of the stimulation amplitude delivered through each of electrodes 112a, 112b will result in similar stimulation to the regions near electrodes 112a, 112b. Shifting 100% of the stimulation amplitude to electrode 112b will effectively rotate the stimulation region 120 degrees relative to the initial stimulation that was delivered only through electrode 112a. Additional examples of moving stimulation around or along the stimulation lead can be found in, for example, U.S. Pat. No. 8,473,061, which is incorporated herein by reference in its entirety.

Instead of, or in addition to, the electrodes 120, 122, a lead 12 can include one or more optical emitters (which can, for example, replace any of the electrodes 120, 122). Each optical emitter can be a light source (for example, a light emitting diode (LED), light emitting transistor (LET), laser diode, a vertical cavity side-emitting laser (VCSEL), an organic light emitting diode (OLED), an organic light emitting transistor (OLET), a lamp, or the like) or can be a light emission region of an optical waveguide (for example, a fiber optic, optical fiber, lens, or any other suitable conveyance of light) or the like. Examples of leads with optical emitters can be found at U.S. Pat. Nos. 9,415,154; 10,335,607; 10,625,072; and 10,814,140 and U.S. Patent Application Publications Nos. 2013/0317572; 2013/0317573; 2017/0259078; 2017/0225007; 2018/0110971; 2018/0369606; 2018/0369607; 2019/0209849; 2019/0209834; 2020/0094047; 2020/0155584; 2020/0376262; 2021/0008388; 2021/0008389; 2021/0016111; and 2022/0072329; all of which are incorporated herein by reference in their entireties.

The following discussion uses electrical stimulation systems with electrodes as examples. It will be understood that the optical stimulation or modulation systems or electrical/optical stimulation systems can be used in place of the example electrical stimulation systems. Thus, a stimulation system can include electrodes, optical emitters, or any combination thereof.

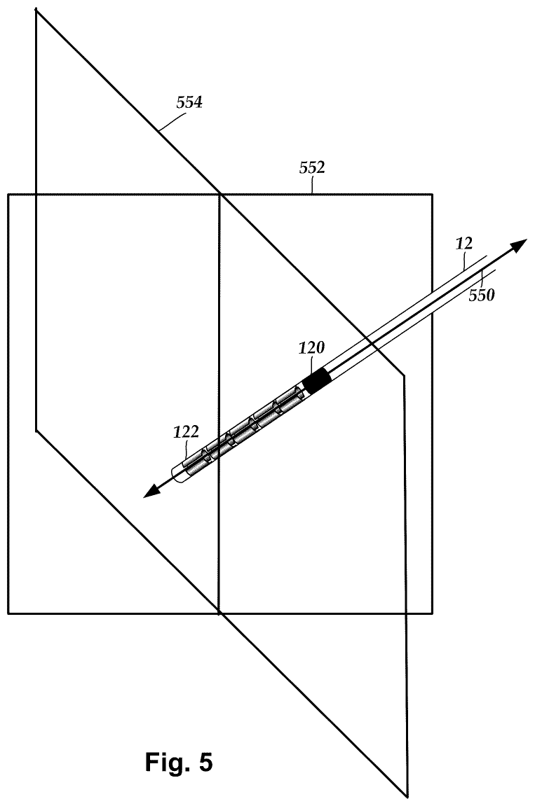

Conventionally, the programming of stimulation and modification of the stimulation parameters to move the stimulation along or around the stimulation lead is conceptualized in the coordinate system of the lead. For example, the stimulation can be shifted up or down the longitudinal axis of the stimulation lead by shifting the stimulation to one or more electrodes at a different electrode level (i.e., longitudinal position) along the lead. The longitudinal axis 550 of the stimulation lead 12 is commonly described as the z-axis of the lead coordinate system (although the longitudinal axis could also be considered the x-axis or y-axis) as illustrated in FIG. 5. The stimulation can be shifted circumferentially around the lead using segmented electrodes, as described above.

Clinicians typically think in terms of anatomically-defined directions (i.e., directions defined by the coronal, sagittal, and transverse planes of the patient), such as anterior, posterior, medial, lateral, superior, or inferior (or combinations of these directions, such as posterior lateral or the like). In many instances, the stimulation lead 12 is inserted into the patient (for example, into the brain of the patient) at a non-zero angle relative to one (or both) of the coronal plane 552 or the sagittal plane 554 of the patient's body. In these instances, the longitudinal axis 550 of the stimulation lead is not parallel to one (or both) of the coronal plane or sagittal plane of the patient.

FIG. 5 illustrates the stimulation lead inserted at a non-zero angle relative to both the coronal plane 552 and the sagittal plate 554. As illustrated in FIG. 5, the coordinate system of the stimulation lead 12 is not aligned with the anatomical coordinate system defined by the coronal, sagittal, and transverse planes.

In at least some instances, the lack of alignment between the longitudinal axis of the stimulation lead and the anatomical coordinate system can make visualization of changes to the stimulation difficult for a clinician that normally think in terms of the anatomically-defined directions. Movement of the stimulation along or around the longitudinal axis of the stimulation lead is not easily translated into the anatomically-defined directions or vice versa. As an example, if a clinician wanted to move the stimulation in an anterior direction using conventional programming device that is programmed in the coordinate system of the lead, the clinician would need to visualize the orientation of the stimulation lead relative to at least the sagittal plane of the patient and determine how an anterior shift in the stimulation would translate to a shift of the stimulation along the stimulation lead in order to select electrode(s) and amplitude(s) that would produce the shifted stimulation. This can be a complex geometrical problem and may require time-consuming calculation.

In contrast to conventional programming interfaces, which are arranged to define or move the stimulation in the coordinate system of the stimulation lead, the systems and methods described herein have an interface and controls that allow the user to move stimulation in anatomically-defined directions (i.e., directions defined by the coronal, sagittal, and transverse planes of the patient or by anatomical directional axes or anatomical directional pairs). Such anatomically-defined directions include one or more the following anatomical directional pairs (which also describe the anatomical directional axes): anterior/posterior, medial/lateral, and superior/inferior or any combination of directions from different directional pairs (for example, posterior lateral). In at least some embodiments, the systems or methods allow the user to move stimulation in directions corresponding to two or more of the directional pairs (for example, anterior/posterior and medial/lateral) or all three directional pairs. In at least some embodiments, the systems or methods allow the user to move stimulation in directions corresponding to a combination of directions from two or more of the directional pairs (for example, posterior lateral).

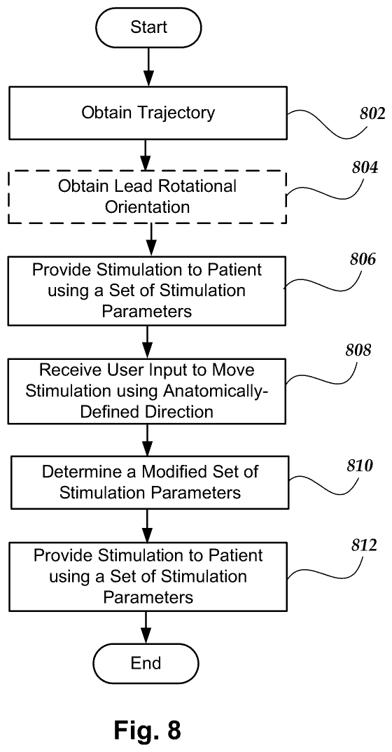

FIG. 8 illustrates one embodiment of a method for moving stimulation using anatomically-defined directions. In step 802, the trajectory of at least the distal end portion (e.g., the portion that contains the electrodes) of the stimulation lead is determined, estimated, or otherwise obtained. In at least some embodiments, the trajectory is defined relative to one, two, or three of the anatomical planes, anatomical direction axes, or anatomical directional pairs. In at least some embodiments, the trajectory can be defined using one, two, or more of the following directional pairs: anterior/posterior, medial/lateral, and superior/inferior (or any other terms or words used to denote anatomical direction). As an example, the trajectory can be input as “posterior” or “posterior-lateral” (or any other variation of these words or similar/equivalent words, terms, or symbols). In at least some embodiments, the trajectory of at least the distal end portion of the stimulation lead can be defined by an angle of the trajectory relative to each of one, two, or three of the sagittal, coronal, or transverse planes. In at least some embodiments, the input of an angle of the trajectory may be limited to increments of 1, 5, 10, or 15 or more degrees (or any other suitable limitation on the angular increments).

In at least some embodiments, the trajectory is provided, determined, estimated, entered, or otherwise obtained by the system or a user (for example, a surgeon, clinician, programmer, or the like). For example, a neurosurgeon or other user can enter a planned trajectory before or after surgery or an actual or estimated trajectory after surgery. As another example, the trajectory of the stimulation lead can be determined using imaging, such as fluoroscopy, CT imaging, or the like. In at least some embodiments, the trajectory may be determined by imaging through observation of electrodes, the lead body, the tip of the stimulation lead, an orientation marker, any other suitable marker, or the like or any combination thereof. In at least some embodiments, a system is capable of receiving one or more images and determining the trajectory of the lead from the images. In at least some embodiments, the determination can be automatic or without assistance from a user. In at least some embodiments, the determination can be made with assistance from a user. In at least some embodiments, a system can include one or more imaging modalities to obtain the image(s). In at least some embodiments, the imaging uses anatomical features, atlas registration, or any other suitable technique to identify the position of at least the distal portion of the lead with respect to anatomical landmarks.

Any other suitable method for determining, estimating, entering, or otherwise obtaining the trajectory can be used. As an example, FIG. 6 illustrates one embodiment of an interface 660 for entering a determined or estimated trajectory. The trajectory can be entered using a trajectory control 662. In at least some embodiments, the trajectory control 662 allow a user to input the direction of the trajectory or to select the direction of the trajectory from a menu. Any other suitable method for inputting a trajectory can be used. Examples of methods, terms, and angle increments for inputting a trajectory are presented above.

In optional step 805, a rotational orientation of the stimulation lead is obtained. In at least some embodiments, particularly for a lead with segmented electrodes, the interface 660 can include a control 654 for entering a rotational orientation of the stimulation lead. Alternatively or additionally, the rotational orientation can be determined or estimated by the system. Non-limiting examples of methods for determining orientation of a stimulation lead through imaging can be found at U.S. Patent Application Publication No. 2018/0104482 and U.S. Pat. Nos. 10,067,659; 10,265,531; and 10,631,932, all of which are incorporated herein by reference in their entireties.

In at least some embodiments, the rotational orientation can be determined or estimated using an orientation marker on the stimulation lead. Non-limiting examples of orientation markers and other arrangements for determining orientation of a stimulation lead can be found at U.S. Pat. Nos. 8,744,596; 8,831,731; 8,831,742; 9,220,889; and 10,525,257, all of which are incorporated herein by reference in their entireties.

In at least some embodiments, the orientation marker is disposed near the electrodes along a distal portion of the stimulation lead. Such an orientation marker may be observed, at least in some embodiments, fluoroscopically or using any other suitable imaging technique.

In at least some embodiments, an orientation marker is disposed along a proximal portion, or at or near the proximal end, of the stimulation lead. Such an orientation marker may be observed, at least in some embodiments, visually, fluoroscopically, or using any other suitable imaging technique.

In step 806, stimulation is provided to the patient using a set of stimulation parameters. Examples of providing stimulation using a set of stimulation parameters can be found in the references cited above.

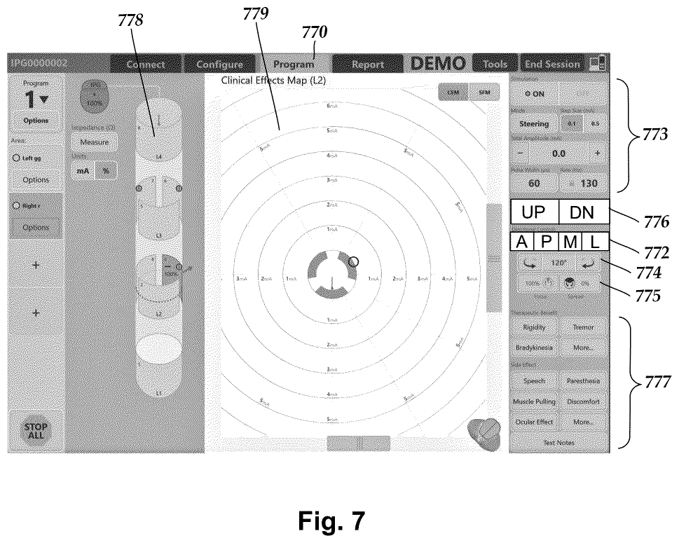

In step 808, a user input to move the stimulation using anatomically-defined direction(s) is received. FIG. 7 illustrates one embodiment of a programming interface 770 that include one or more directional controls 772 for moving the stimulation in anatomically-defined direction(s) such as Anterior (A), Posterior (P), Medial (M), or Lateral (L). In at least some embodiments, the programming interface 770 can include lead directional controls, such as rotational controls 774 for rotating the stimulation around the lead (for example, clockwise or counterclockwise), focus/spread controls 775 to narrow or expand the circumferential extent of the stimulation, or up/down controls 776 to move the stimulation proximally or distally, respectively, along the longitudinal axis of the lead. In at least some embodiments, instead of including all of these controls, the programming interface 770 can include a direction selection control that can be used to present either the directional controls 772 or the up/down controls 776 (and optionally one or both of the rotational controls 774 or focus/spread controls 775).

The programming interface 770 can include parameter controls 773 for modifying other stimulation parameters (such as amplitude, pulse width, pulse rate, or the like), effect controls 777 for inputting information about therapeutic benefits or side effects, a representation 778 of the stimulation lead and electrodes with the active electrodes highlighted (and optionally including the electrode fractionalization), a clinical effects map 779, or the like or any combination thereof. In at least some embodiments, instead of a clinical effects map 779, the programming interface 770 can include an estimated volume of activation which indicates which region of tissue is estimated to be stimulated above a threshold level for the selected stimulation parameters. Any suitable arrangement or method for determining and displaying the estimated volume of activation (or similar element such as an stimulation field volume, volume of tissue activated, or the like) can be used including, but not limited to, those described in, for example, U.S. Pat. Nos. 8,326,433; 8,675,945; 8,831,731; 8,849,632; 8,958,615; 10,603,498; 10,780,282; 10,814,140; 11,285,329; and 11,357,986 and U.S. Patent Application Publications Nos. 2009/0287272; 2009/0287273; 2012/0314924; 2013/0116744; 2014/0122379; 2015/0066111; and 2019/0015660, all of which are incorporated herein by reference.

In at least some embodiments, instead of a clinical effects map 779, the programming interface can include a representation of the lead and, optionally, specific anatomical regions around the lead. The representation may be two-dimensional or three-dimensional. In at least some of these embodiments, an estimated volume of activation can be disposed around or adjacent the representation of the lead to indicate which region of tissue is estimated to be stimulated above a threshold level for the selected stimulation parameters. In at least some embodiments, as the user changes the stimulation parameters or uses the directional controls 772 (and optionally the rotational controls 774, focus/spread controls 775, or up/down controls 776) to move the stimulation, the estimated volume of activation is modified to represent the modified stimulation.

In step 810, a modified set of stimulation parameters is determined. With the trajectory of the stimulation lead (and, optionally, the rotational orientation of the stimulation lead) known, a system can transform directions from the anatomically-defined coordinate system/directions to the lead coordinate system/directions (or from the lead coordinate system/directions to the anatomically-defined coordinate system/directions, if desired). A user can direct the system to move stimulation using anatomically-defined direction(s). The system can then translate these anatomically-defined direction(s) to changes in electrode selection, electrode fractionalization, or other stimulation parameters (or any combination thereof) to achieve the desired movement. In at least some embodiments, the system can perform a coordinate transformation from the anatomically-defined coordinate system/directions to the lead coordinate system/directions. In at least some embodiments, the system can utilize an algorithm or other computational method to determine the changes in electrode selection, electrode fractionalization, or other stimulation parameters (or any combination thereof) to achieve the requested movement of the stimulation.

In step 812, stimulation is provided to the patient using the modified set of stimulation parameters. Steps 808 to 812 can be repeated with each new user input to move the stimulation.

It will be understood that each block of the flowchart illustration, and combinations of blocks in the flowchart illustration and methods disclosed herein, can be implemented by computer program instructions. These program instructions may be provided to a processor to produce a machine or engine, such that the instructions, which execute on the processor, create means for implementing the actions specified in the flowchart block or blocks or engine disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computing device. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device. The computer program instructions can be stored locally or nonlocally (for example, in the Cloud).

The above specification and examples provide a description of the arrangement and use of the invention. Since many embodiments of the invention can be made without departing from the spirit and scope of the invention, the invention also resides in the claims hereinafter appended.

1. A method for modifying stimulation by a stimulation system comprising a stimulation device and a stimulation lead coupled to the stimulation device and comprising a distal end portion implanted in a patient and a plurality of electrodes, optical emitters, or any combination thereof disposed along the distal end portion, wherein the distal end portion is not parallel to at least one of a sagittal plane or a coronal plane of the patient, the method comprising:
providing stimulation to the patient by the stimulation device through at least one of the electrodes or optical emitters of the stimulation lead according to an initial set of stimulation parameters;
receiving, at a programming device, a user input to move the stimulation in a selected anatomically-defined direction;
determining, by the programming device or the stimulation device, a modified set of stimulation parameters that moves the stimulation in the selected anatomically-defined direction; and
providing stimulation to the patient by the stimulation device through at least one of the electrodes or optical emitters of the stimulation lead according to the modified set of stimulation parameters.

2. The method of claim 1, wherein the anatomically-defined direction is selected from anterior, posterior, superior, inferior, lateral, medial, or any combination thereof.

3. The method of claim 1, further comprising obtaining a trajectory of the distal end portion of the stimulation lead.

4. The method of claim 3, wherein obtaining the trajectory comprises receiving, at the programming device, a user input of the trajectory.

5. The method of claim 3, wherein obtaining the trajectory comprises determining an actual or estimated trajectory from imaging.

6. The method of claim 1, further comprising obtaining a rotational orientation of the stimulation lead.

7. The method of claim 6, wherein obtaining the rotational orientation comprises observing an orientation marker of the stimulation lead or estimating the rotational orientation from responses to stimulation.

8. The method of claim 6, wherein obtaining the rotational orientation comprises determining the rotational orientation from imaging of the stimulation lead.

9. The method of claim 1, wherein determining the modified set of stimulation parameters comprises transforming the selected anatomically-defined direction into a lead coordinate system or at least one lead direction.

10. The method of claim 1, wherein determining the modified set of stimulation parameters comprises determining the modified set of stimulation parameters using a predefined algorithm with the selected anatomically-defined direction being an input to the algorithm.

11. The method of claim 1, wherein the programming device comprises a control for switching between user input controls for moving stimulation along anatomically-defined directions and user input controls for moving stimulation along lead directions.

12. A programming device for modifying stimulation by a stimulation system comprising a stimulation device and a stimulation lead coupled to the stimulation device and comprising a distal end portion implanted in a patient and a plurality of electrodes or optical emitters disposed along the distal end portion, wherein the distal end portion is not parallel to at least one of a sagittal plane or a coronal plane of the patient, the system comprising:
at least one processor configured to perform actions, the actions comprising:
receiving a user input to move stimulation in a selected anatomically-defined direction;
determining a modified set of stimulation parameters that moves the stimulation in the selected anatomically-defined direction; and
transmitting the modified set of stimulation parameters to the stimulation device.

13. The programming device of claim 12, wherein the anatomically-defined direction is selected from anterior, posterior, superior, inferior, lateral, medial, or any combination thereof.

14. The programming device of claim 12, wherein the actions further comprise obtaining a trajectory of the distal end portion of the stimulation lead.

15. The programming device of claim 14, wherein obtaining the trajectory comprises receiving, at the programming device, a user input of the trajectory.

16. The programming device of claim 12, wherein the actions further comprise obtaining a rotational orientation of the stimulation lead.

17. The programming device of claim 12, wherein determining the modified set of stimulation parameters comprises transforming the selected anatomically-defined direction into a lead coordinate system or at least one lead direction.

18. The programming device of claim 12, wherein determining the modified set of stimulation parameters comprises determining the modified set of stimulation parameters using a predefined algorithm with the selected anatomically-defined direction being an input to the algorithm.

19. The programming device of claim 12, wherein the programming device comprises a control for switching between user input controls for moving stimulation along anatomically-defined directions and user input controls for moving stimulation along lead directions.

20. A stimulation system, comprising:
the programming device of claim 12;
the stimulation lead comprising the plurality of electrodes, optical emitters, or any combination thereof; and
the stimulation device coupled or coupleable to the stimulation lead, wherein the stimulation device comprises a processor configured to perform actions, the actions comprising
providing stimulation to the patient through at least one of the electrodes or optical emitters of the stimulation lead according to an initial set of stimulation parameters, and
after the modified set of stimulation parameters is transmitted to the stimulation device, providing stimulation to the patient through at least one of the electrodes or optical emitters of the stimulation lead according to the modified set of stimulation parameters.
